(19) United States
(12) Patent Application Publication
(10) Pub. No.: US 2024/0285925 A1
(43) Pub. Date: Aug. 29, 2024

(54) Optimized Port Stem Geometry

(71) Applicant: Bard Peripheral Vascular, Inc., Franklin Lakes, NJ (US)

(72) Inventors: Christian Andersen, Queen Creek, AZ (US); Diana Fiumefreddo, Salt Lake City, UT (US); Ian N. Thomas, West Bountiful, UT (US); Jessica Hoye, Phoenix, AZ (US); Bryon Ray Densley, Rochester, MN (US)

(21) Appl. No.: 18/575,771

(22) PCT Filed: Jul. 7, 2021
(86) PCT No.: PCT/US2021/040715

§ 371 Date: Dec. 29, 2023

(51) Int. Cl. A61M 39/12; A61M 39/02; A61M 39/10

(52) U.S. Cl. CPC A61M 39/12; A61M 39/0208; A61M 2039/1027; A61M 2205/04

(57) ABSTRACT

Embodiments disclosed herein are directed to a stem including optimized geometry configured to maximize fluid flow while facilitating engagement with a lumen of a catheter. The stem can define a lumen having a distal opening defining a first diameter. The tip structure can define a tip structure lumen having a second diameter that is less than the first diameter. The tip structure can include one or more fingers extending longitudinally distally and defining one or more slots. The tip structure can fit within the catheter lumen, facilitating engagement therewith, while the one or more slots can improve fluid flow, providing little or no reduction in fluid flow relative to the distal opening.

### SUMMARY

Briefly summarized, embodiments disclosed herein are directed to a stem configured to provide fluid communication between a catheter and a medical device (e.g. a port). The stem includes optimized geometry configured to maximize fluid flow through the stem lumen, while facilitating engagement with a lumen of the catheter.

Coupling catheters, or similar compliant tubular devices, with rigid stems, e.g. a port stem or the like, can be a challenging process especially within the confined, wetted environment of subcutaneous placements. Typically, the stem is urged into the lumen of the catheter, and the compliant catheter elastically deforms to engage the stem in an interference fit. Optionally a cathlock can further secure the catheter to the port stem to prevent fluid leakage. The relative sizes of the inner diameter of the catheter lumen, and the outer diameter of the stem can be critical. Where a stem tip outer diameter is too large, coupling the catheter to the stem can be challenging, leading to kinking or collapsing of the catheter. Where the stem tip outer diameter is too small, a corresponding inner diameter of the stem lumen restricts fluid flow therethrough. Further, thinner catheters (i.e. lower French size catheters) have relatively thinner walls, making the catheter more susceptible to kinking or collapse.

Disclosed herein is a stem for providing fluid communication between an access port and a catheter, the stem including an elongate body defining a body lumen, a distal opening of the body lumen having a first diameter, and a tip structure extending distally from the distal opening and having a tip lumen having a second diameter smaller than the first diameter, and a plurality of slots extending from a distal end of the tip structure to the distal opening of the body lumen.

In some embodiments, the plurality of slots define a plurality of fingers. In some embodiments, the plurality of fingers taper from a first wall thickness at a proximal end to a second wall thickness, less than the first wall thickness, at a distal end. In some embodiments, the tip structure comprises an outer surface that tapers distally from a first larger diameter to a second smaller diameter. In some embodiments, the elongate body comprises a circumferential ridge disposed along an outer surface of the elongate body.

In some embodiments, the stem is coupled to the access port with one of an interference fit, press-fit, or snap-fit engagement. In some embodiments, the slot extends through an arc distance of less than 350°. In some embodiments, a finger of the plurality of fingers extends radially through an arc distance of less than 350°.

Also disclosed is a stem for providing fluid communication between a medical device and a catheter including, a body defining a lumen extending between a proximal opening and a distal opening, the distal opening defining a first diameter, a tip structure disposed at a distal end of the body having, one or more fingers extending radially inwards and distally from the distal opening, an inner surface of a first finger of the one or more fingers extending along an axis disposed radially inward from the distal opening.

In some embodiments, the first finger extends radially through an arc distance of less than 350°. In some embodiments, the finger includes a proximal portion extending from the distal opening radially inward and distally, and a distal portion supported by the proximal portion and extending distally therefrom. In some embodiments, an inner surface of the distal portion extends parallel to the longitudinal axis and an outer surface of the distal portion extends at an angle relative to the longitudinal axis.

In some embodiments, the stem further includes a second finger of the plurality of fingers and disposed at a different radial position about the axis of the lumen, an inner surface of the first finger and an inner surface of the second finger defining a second diameter that is less than the first diameter. In some embodiments, an outer surface of the first finger and an outer surface of the second finger defines a tapered outer profile extending from the second diameter to the first diameter. In some embodiments, the first finger and the second finger define a slot extending longitudinally therebetween to the distal opening. In some embodiments, the slot extends radially through an arc distance of less than 350°.

Also disclosed is a method of coupling a catheter with a medical device including, providing a stem formed of a rigid material and defining a lumen extending between a distal opening and a proximal opening, the distal opening defining a first diameter, the stem having a distal tip structure including two or more fingers extending longitudinally distally from the distal opening, distal tip of the two or more fingers co-operating to form a second diameter, less than the first diameter, providing a catheter formed of a compliant material and defining a lumen having a third diameter that is larger than the second diameter, urging the tip structure into the catheter lumen, and elastically deforming the catheter from the third diameter to the first diameter.

In some embodiments, the third diameter of the catheter lumen is equal to or less than the first diameter of the distal opening. In some embodiments, a finger of the two or more fingers includes a proximal portion extending radially inward from the distal opening, and a distal portion extending longitudinally distally from the proximal portion and defining the inner surface. In some embodiments, an outer surface of the two or more fingers defines a tapered profile extending proximally from the second diameter to the first diameter. In some embodiments, the two or more fingers define a slot extending longitudinally therebetween from a distal tip of the tip structure and the distal opening.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 shows a perspective view of a stem including optimized tip geometry in an exemplary environment of use, in accordance with embodiments disclosed herein.

FIG. 2A shows a perspective view of a stem including optimized tip geometry, in accordance with embodiments disclosed herein.

FIG. 2B shows a side view of a stem including optimized tip geometry, in accordance with embodiments disclosed herein.

FIG. 2C shows a longitudinal cross-section view of a stem including optimized tip geometry, in accordance with embodiments disclosed herein.

FIG. 2D shows close up detail of the stem of FIG. 2C, in accordance with embodiments disclosed herein.

FIG. 3A shows a distal end view of a stem, in accordance with embodiments disclosed herein.

FIG. 3B shows a distal end view of a stem, in accordance with embodiments disclosed herein.

FIG. 3C shows a longitudinal cross-section view of a stem, in accordance with embodiments disclosed herein.

FIG. 3D shows a distal end view of the stem of FIG. 3C, in accordance with embodiments disclosed herein.

FIGS. 4A-4B show an exemplary method of use of a stem including optimized tip geometry, in accordance with embodiments disclosed herein.

FIG. 5A shows close up detail of a longitudinal cross-section of a stem, in accordance with embodiments disclosed herein.

FIG. 5B shows a lateral cross-section view of the stem of FIG. 5A, in accordance with embodiments disclosed herein.

FIG. 5C shows close up detail of a longitudinal cross-section of a stem, in accordance with embodiments disclosed herein.

FIG. 5D shows a lateral cross-section view of the stem of FIG. 5C, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

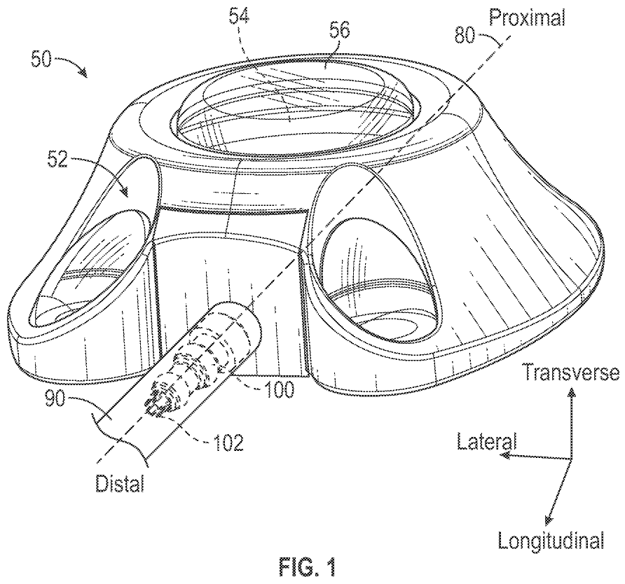

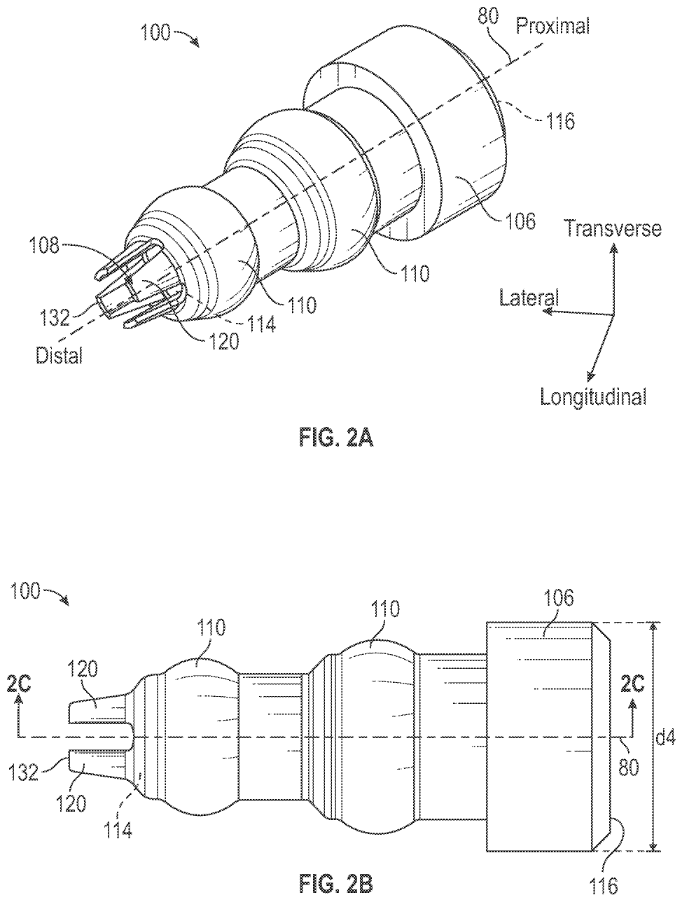

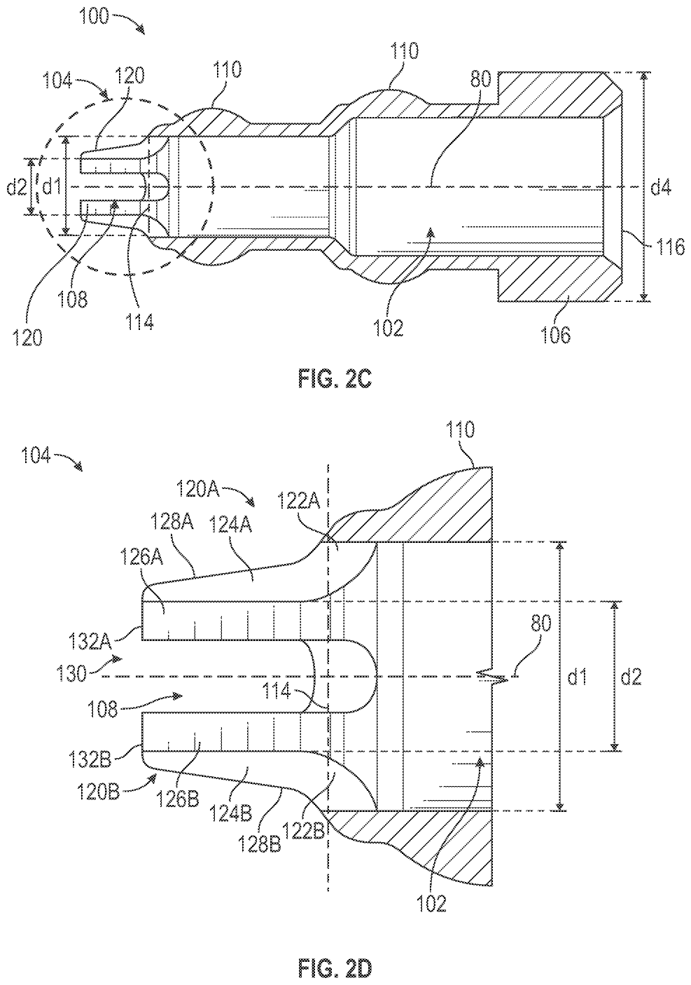

To assist in the description of embodiments described herein, as shown in FIGS. 1-2A, a longitudinal axis extends substantially parallel to an axial length of the stem 100. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

Embodiments described herein are directed to a stem 100 including tip geometry configured to facilitate coupling a catheter 90, or similar compliant tube, with the stem 100 while maximizing fluid flow therethrough. FIG. 1 shows a stem 100 including optimized geometry in an exemplary environment of use. In an embodiment, the stem 100 can be configured to provide fluid communication between a catheter 90 and a medical device, such as a port 50. The port 50 can generally include a body 52 defining a reservoir 54 that is in fluid communication with a lumen 102 of the stem 100. The port 50 can further include a needle penetrable septum 56 disposed over the reservoir and configured to provide access thereto. In use, an access needle can extend percutaneously, through the needle-penetrable septum 56 and into the reservoir 54 to provide fluid communication therewith. As will be appreciated, the port 50 is a non-limiting, exemplary medical device, and embodiments disclosed herein can be used with various similar medical devices that include stems or similar structures.

The catheter 90 can include an elongate tube extending longitudinally and defining a catheter lumen 92. In an embodiment, a distal tip of the catheter 90 can be disposed within a vasculature of the patient to provide fluid communication therewith. A proximal end of the catheter 90 can be coupled with the stem 100, as described in more detail herein. It will be appreciated that the catheter 90 is not intended to be limiting, and that embodiments disclosed herein can be used with various compliant, tubular devices configured to provide fluid communication. The catheter 90 can be formed of a compliant material such as a plastic, polymer, elastomer, composite, or the like. A proximal end of the catheter 90 can be configured to elastically deform and stretched over the stem 100 to provide a fluid tight seal therebetween. As will be appreciated, the exemplary environment of use is not intended to be limiting and embodiments described herein can be used with various medical tube couplings that include a compliant tube coupling with a rigid structure and require a fluid tight seal therebetween while maximizing fluid throughflow.

FIGS. 2A-2D show further details of an embodiment of the stem 100. FIG. 2A shows a perspective view of the stem 100. FIG. 2B shows a side view of the stem 100. FIG. 2C shows a longitudinal cross-section view of the stem 100. FIG. 2D shows close up detail of the distal tip structure 104 of the stem 100. In an embodiment, the stem 100 can be formed integrally with the medical device, e.g. port 50. In an embodiment, the stem 100 can be formed as a separate structure, (e.g. as shown in FIG. 2A) and can be coupled with the medical device using a press fit, snap-fit, interference fit engagement, and/or with adhesive, bonding, welding, or the like. In an embodiment, the stem 100 can define a substantially rigid structure formed from a plastic, polymer, metal, alloy, composite, or similar suitable material. In an embodiment, the stem 100 can define a radially symmetrical structure extending about a central axis 80, which extends parallel to the longitudinal axis.

As shown in FIGS. 1 and 2C, in an embodiment, the stem 100 can define a lumen 102 extending longitudinally from a distal opening 114 to a proximal opening 116. In an embodiment, the distal opening 114 can define a first diameter (d1). As shown in FIG. 2C, the stem 100 can further include a tip structure 104 extending distally from the distal opening 114 and defining a tip lumen 108 having a second diameter (d2) which is less than the first diameter (d1) of the distal opening 114. In an embodiment, the inner diameter of the catheter lumen 92 in a relaxed state can define a third diameter (d3). In an embodiment, the catheter lumen diameter (d3) can be equal to or greater than the second diameter (d2), as described in more detail herein. In an embodiment, the catheter lumen 92 can elastically deform to a diameter larger than the catheter lumen diameter (d3) of the relaxed configuration.

As shown in FIGS. 2A-2C, in an embodiment, an outer profile of the stem 100 can mirror the inner profile of the lumen 102. In an embodiment, the outer profile of the stem 100 can define a continuous or discontinuous, cylindrical or tapered shape or can include combinations thereof. In an embodiment, the outer profile of the stem 100 can include one or more transition portions defining a change in outer diameter between one or more outer diameters of the stem 100. The transition portion can include a tapered or stepped transition between the different diameters. A stepped transition portion can include a portion that extends substantially perpendicular to the longitudinal axis.

In an embodiment, the stem 100 can include one or more ridges 110 extending circumferentially about the central axis 80 and disposed along an outer surface of the stem 100. The ridge 110 can define a rounded, triangular, stepped, or barbed longitudinal profile and define an increase in outer diameter of the stem 100. The ridge 110 can be configured to increase friction between the stem 110 and a catheter 90, when engaged therewith, to facilitate gripping the catheter 90 in an interference fit.

In an embodiment, the ridge 110 can be configured to facilitate coupling a cathlock with the stem 100 and catheter 90 assembly. For example, with the catheter 90 engaged with an outer surface of the stem 100, a cathlock can engage an outer surface of a portion of the catheter 90 and compress the catheter 90 thereto, further securing the catheter 90 thereto. The cathlock can include a corresponding abutment extending radially inward and configured to engage the ridge 110, optionally through the catheter 90, in a snap-fit or interference fit engagement. In an embodiment, the cathlock can engage the port 50 in a press-fit, snap-fit, or interference fit engagement.

In an embodiment, the stem 100 can include a proximal engagement portion 106 disposed adjacent the proximal opening 116. The engagement portion 106 can define a substantially cylindrical shape, extending longitudinally and defining a diameter (d4). The engagement portion 106 can be configured to engage the port 50 in a press-fit or interference-fit engagement. In an embodiment, the engagement portion 106 can include one or more protrusions, detents, pawls, ridges, barbs, or the like, configured to engage the port 50 in a snap-fit engagement. A proximal rim of the engagement portion 106 can define a beveled or chamfered edge to facilitate engagement with the port 50. In an embodiment, a diameter (d4) of the engagement portion 106 can be the same or slightly larger than a diameter of a stem receiving recess disposed in the port 50.

As shown in FIGS. 2C-2D, in an embodiment, the stem lumen 102 can define a cylindrical profile extending between the distal opening 114 and the proximal opening 116, i.e. a wall of the lumen 102 can extend parallel to a longitudinal axis. In an embodiment, the lumen 102 can define a continuous diameter along the longitudinal length thereof, e.g. a first diameter (d1). In an embodiment, the stem lumen 102 can define a tapered profile extending between distal opening 114 and the proximal opening 116, i.e. a wall of the lumen 102 can extend at an angle relative to the longitudinal axis. In an embodiment, the lumen 102 can define a continuous change in diameter along the longitudinal length thereof. In an embodiment, the inner diameter of the proximal opening 116 can be larger than the inner diameter of the distal opening 114, e.g. the first diameter (d1).

In an embodiment, the stem lumen 102 can define a discontinuous change in diameter along the longitudinal length thereof. For example, as shown in FIG. 2D, the lumen 102 can include a first portion defining a first diameter (d1) disposed adjacent the distal opening 114. The lumen 102 can further include one or more second portions defining a different diameter from that of the first diameter (d1) of the first portion. The diameter of the one or more second portions can be greater than, or smaller than, the first diameter (d1). Further, the lumen 102 can include one or more transition portions disposed between the first portion defining the first diameter (d1), and the one or more second portions defining one or more different diameters. The transition portion can either be tapered, i.e. a wall thereof extending at an angle relative to the longitudinal axis, or be stepped, i.e. a wall thereof extending perpendicular relative to the longitudinal axis.

In an embodiment, the proximal opening 116 can include a beveled edge configured to facilitate a fluid flow into, or out of, the lumen 102 and minimize fluid flow turbulence. In an embodiment, a diameter of the proximal opening 116 can be the same as, or different from the diameter (d1) of the distal opening 114. In an embodiment, the diameter of the proximal opening 116 can be larger or smaller than the first diameter (d1) of the distal opening 114.

As shown in FIGS. 2C-2D, in an embodiment, the distal opening 114 can define a continuous inner surface extending annularly about the axis 80 of the lumen 102 and define a first diameter (d1). In an embodiment, the stem 100 can include a tip structure 104 extending distally from the distal opening 114 of the stem 100. In an embodiment, the tip structure 104 can define a tip lumen 108 having a discontinuous inner surface extending annularly about the axis 80 of the lumen 102 and define a second diameter (d2). In an embodiment, the tip structure 104 can include one or more fingers 120 extending distally from a distal edge of the distal opening 114.

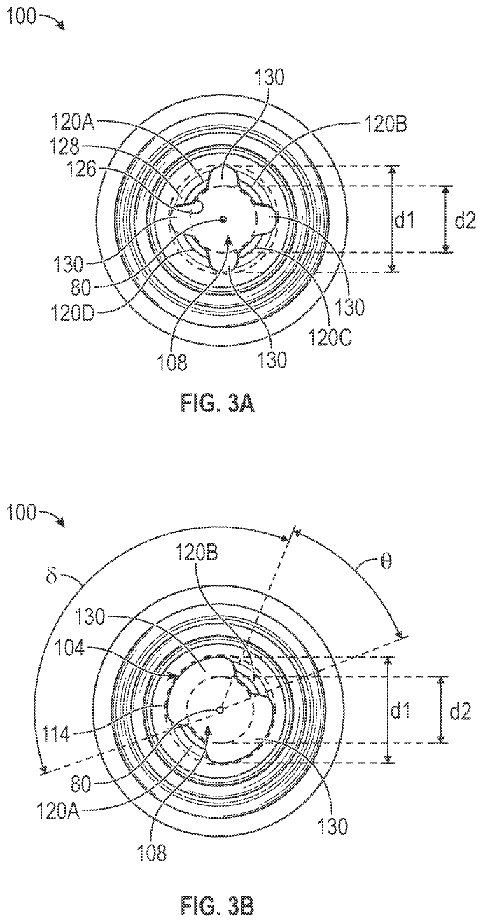

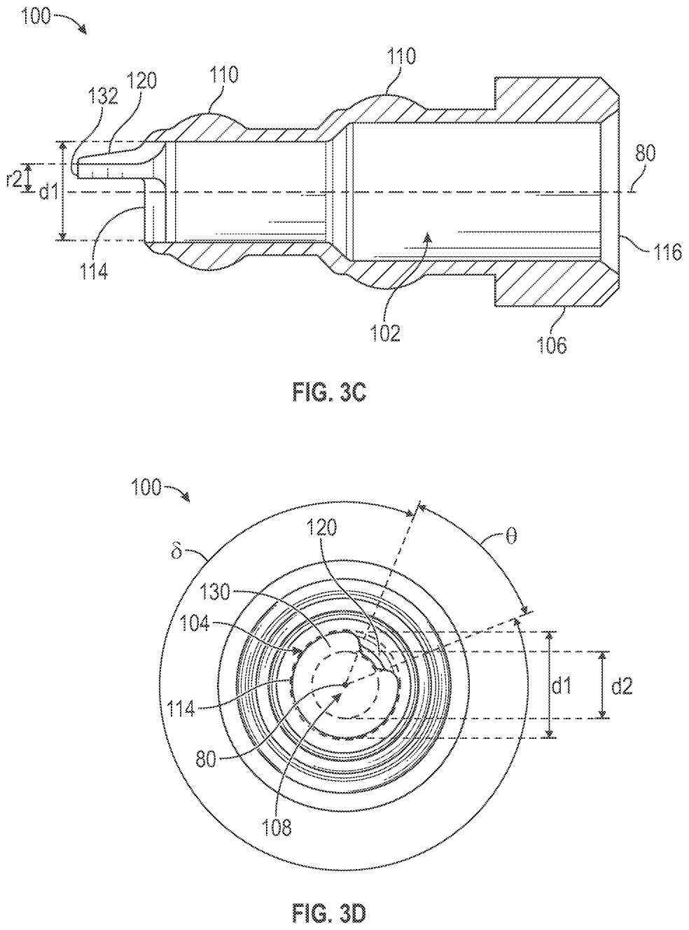

FIGS. 3A-3D show various configurations of tip structure 104 including one or more fingers 120. In an embodiment, the one or more fingers 120 can be disposed radially equally about the distal opening 114. In an embodiment, the one or more fingers 120 can be disposed radially unequally about the distal opening 114. In an embodiment, as shown in FIG. 3A, the tip structure 104 can include four fingers 120, i.e. a first finger 120A, a second finger 120B, a third finger 120C and a fourth finger 120D disposed radially equally about the distal opening 114. In an embodiment, as shown in FIG. 3B, the tip structure 104 can include two fingers 120, i.e. a first finger 120A and a second finger 120B, disposed radially about the distal opening 114. For example, a first finger 120A can be disposed opposite a second finger 120B across the central axis 80. In an embodiment, as shown in FIGS. 3C-3D, the tip structure 104 can include a single finger 120, as described in more detail herein. It will be appreciated, however, that greater or fewer fingers 120 are also contemplated.

In an embodiment, as shown in FIGS. 3B and 3D, each finger 120 can extend radially through an arc distance (O)). In an embodiment, the arc distance (e) can be less than 360º. In an embodiment, the arc distance (O)) can be between 5° and 180º. In an embodiment, the arc distance (e) can be between 35° and 55°. In an embodiment, the tip structure 104 can include a slot 130 extending longitudinally from a distal end of the tip structure 104. In an embodiment, two or more fingers 120 can co-operate to define a slot 130 extending longitudinally from a distal end of the tip structure 104 to the distal opening 114. In an embodiment, as shown in FIG. 3B, the slot 130 can extend radially through an arc distance (δ). In an embodiment, the arc distance (δ) can be less than 360°. In an embodiment, the arc distance (δ) can be between 5° and 180°. In an embodiment, the arc distance (δ) can be between 125° and 145°.

As shown in FIG. 2D, in an embodiment, a finger, e.g. a first finger 120A, of the one or more fingers 120 can include a distal portion 124 extending longitudinally and supported by a proximal portion 122. The proximal portion 122 can extend from a distal edge of the distal opening 114 or from an inner surface of the distal opening 114 and can extend at an angle, radially inward and distally, therefrom. The distal portion 124 can be supported by the proximal portion 122 and can extend longitudinally distally therefrom. An inner surface 126 of the distal portion 124 can extend substantially parallel with a longitudinal axis. An outer surface 128 of the distal portion 124 can extend at an angle relative to the longitudinal axis. Worded differently, the distal portion 124 can define a first wall thickness adjacent the proximal portion 122 and a second wall thickness, less than the first wall thickness, at a distal tip 132 of the finger 120.

In an embodiment, an inner surface 126 of the one or more fingers 120, e.g. a first inner surface 126A and a second inner surface 126B, can co-operate to define a lumen 108 of the tip structure 104 defining a second diameter (d2). In an embodiment, a distal tip 132 of the one or more fingers 120 can co-operate to define a second diameter (d2). In an embodiment, the second diameter (d2) can be less than the first diameter (d1). As shown in FIG. 2D, in an embodiment, an outer surface 128 of the one or more fingers 120, e.g. a first outer surface 128A and a second outer surface 128B, can co-operate to define a tapered outer profile extending longitudinally from a distal tip 132 of the finger 120.

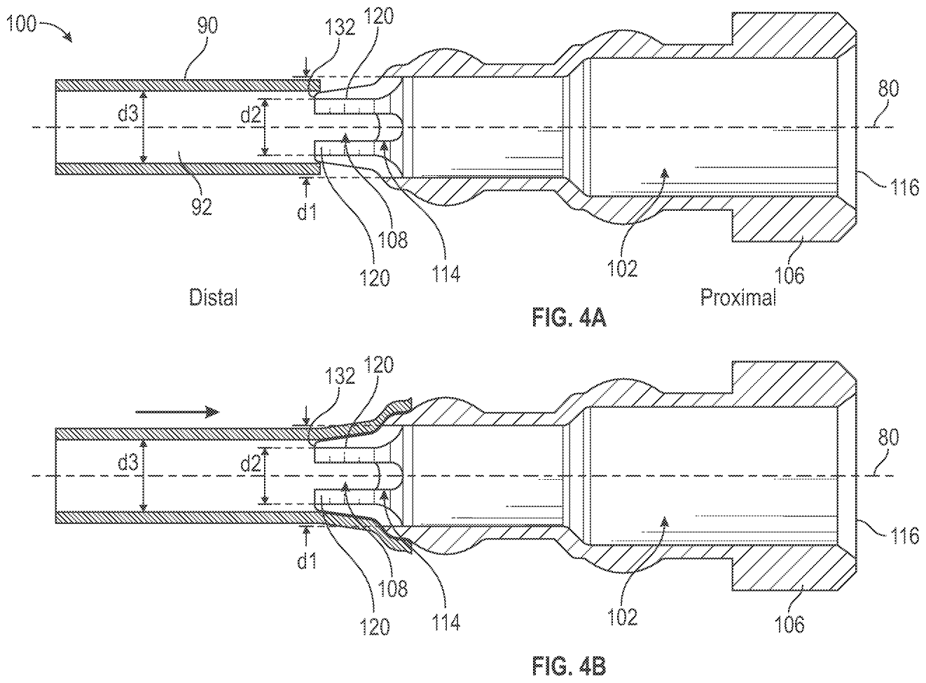

As shown in FIGS. 4A-4B, the distal tip structure 104 can be configured to facilitate coupling the catheter 90 with the stem 100 while maximizing a fluid flow rate through the stem lumen 102. As shown in FIG. 4A, a catheter 90 can define a lumen 92 having a catheter lumen diameter (d3). The catheter lumen diameter (d3) can be equal to or larger than the second diameter (d2) defined by the one or more fingers 120 of the tip structure 104. In an embodiment, the catheter lumen diameter (d3) can be equal to or less than the diameter (d1) of the distal opening 114. The tip structure 104 can be configured to fit within the catheter lumen 92. As shown in FIG. 4B, as catheter 90 is urged proximally, the tapered outer profile of the tip structure 104 can elastically deform the catheter 90 from the catheter lumen diameter (d3) to the first diameter (d1) of the distal opening 114, to fit over the stem 100, creating a fluid tight seal therebetween.

Advantageously, embodiments of the tip structure 104 can engage an inner surface of the catheter lumen 92, to facilitate coupling the catheter 90 with the stem 100, while mitigating any reduction in fluid flow relative to the diameter (d1) of the distal opening 114. For example, as shown in FIG. 3A, the diameter (d1) of the distal opening 114 can provide a first cross-sectional area and, as such, a first fluid flow rate therethrough. The tip structure 104 can define a second diameter (d2) and can fit within the catheter lumen diameter (d3). However, the relatively smaller diameter (d2) of the tip structure 104 can also provide a second, smaller, cross-sectional area, and a second, lower, fluid flow rate therethrough, relative to the diameter (d1) of the distal opening 114. However, the tip structure 104 can also include a slot 130 that can provide an increase in cross-sectional area, relative to the second diameter (d2) alone, mitigating the reduction in fluid flow between the distal opening 114 and the tip structure 104.

In an embodiment, as shown in FIG. 3A, a tip structure 104 can include two or more fingers 120 defining two or more slots 130. As such, the tip structure 104 including two or more slots 130 provides yet further increase in cross-sectional area in addition to the second diameter (d2) and further reduces the impact on fluid flow through the distal opening 114. In an embodiment, as shown in FIG. 3B, a decrease in arc distance (O) of the one or more fingers 120 and thereby an increase in arc distance (δ) of one or more slots 130 provides yet further increase in cross-sectional area in addition to the second diameter (d2) and further reduces the impact on fluid flow through the distal opening 114 at the first diameter (d1).

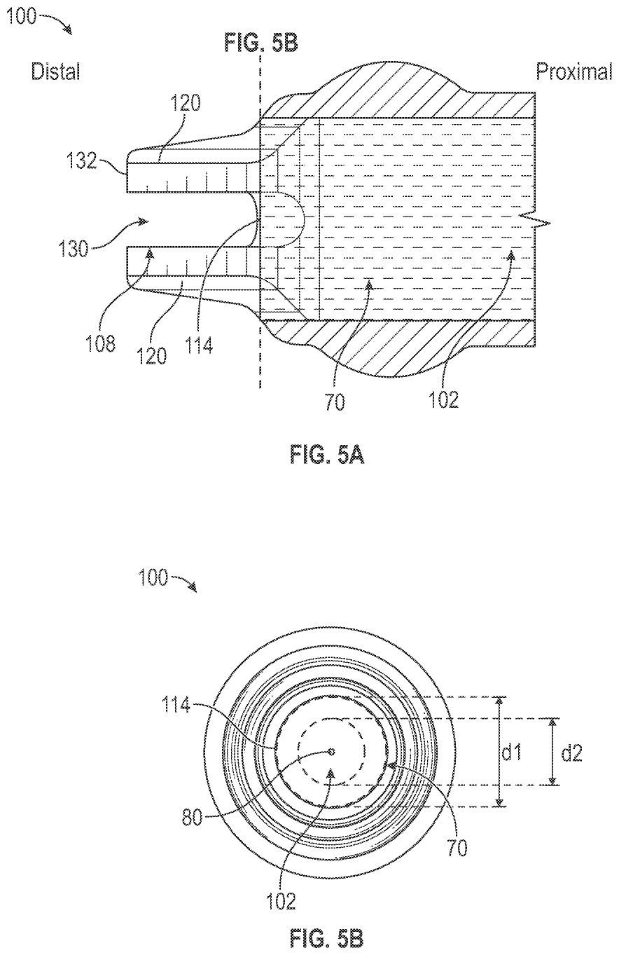

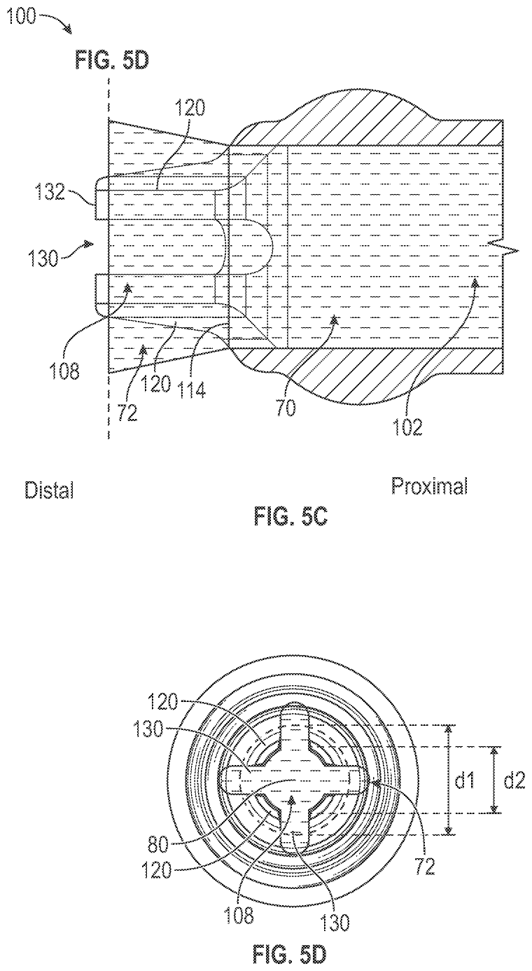

In an embodiment, as shown in FIGS. 5A-5D, the tip structure 104 including the one or more slots 130 can further reduce the impact on fluid flow through the distal opening 114 by allowing fluid, displaced by the finger 120, to flow radially outwards. As shown in FIGS. 5A-5B, the stem lumen 102 extending distally up to the distal opening 114 at a first diameter (d1) provides a first cross sectional area (FIG. 5B) and a first fluid flow 70 therethrough.

As shown in FIGS. 5C-5D, the tip structure 104 defines a lumen 108 having a discontinuous inner surface extending annularly, i.e. the tip structure includes one or more slots 130. As such, even though a finger structure 120 extends radially inward reducing the cross-sectional area through an arc distance (θ), the slots 130 allow the fluid displaced by the finger 120 to flow radially outward providing a second fluid flow 72. In an embodiment, the second fluid flow 72 can expand radially outward to an inner diameter (d3) of the catheter lumen. In an embodiment, the inner diameter (d3) of the catheter lumen can be equal to or greater than the first diameter (d1) of the distal opening. As shown in FIG. 5D, in an embodiment, the second fluid flow 72 can flow radially outward to a larger diameter than the first diameter (d1). As such the drop in cross-section area through the tip structure 104 from the first diameter (d1) to the second diameter (d2) is yet further offset by the radially outward flow 72 through the slots 130.

In an embodiment, the cross-sectional area of the second fluid flow 72 through the distal tip structure 104 (FIG. 5D) can be equal to or greater than the cross-sectional area of the first fluid flow 70 through the distal opening 114 (FIG. 5B). As such, the distal tip structure 104 can provide little to no impact on fluid flow through the stem 100 while still facilitating coupling the stem 100 with the catheter 90.

In an embodiment, a catheter lumen diameter (d3) can be larger than a first diameter (d1) of the distal opening 114. As such, when engaging the stem 100 with the catheter lumen 92, the distal tip structure 104 can extend into the catheter lumen 92 without the catheter 90 elastically deforming. When the catheter lumen 92 engages the outer surface of the stem 100, i.e. at a point where the outer surface of the stem 100 matches the inner lumen diameter (d3), an axial force is required to elastically deform the catheter 90 to fit over the stem 100 and engage therewith in an interference fit. Advantageously, the tip structure 104 extending into the catheter lumen 92 can provide columnar support to the compliant catheter 90, as the axial force is applied. As such, the distal tip structure 104 can mitigate kinking of the catheter 90, facilitating engagement therewith, while have little to no impact of fluid throughflow, as described herein.

In an embodiment, as shown in FIGS. 3C-3D, a tip structure 104 can include a single finger 120 extending distally. An axis of the inner surface 126 of the finger 120 can be disposed radially inward relative to an edge of the distal opening 114. In an embodiment, the inner surface 126 of the finger 120 can be disposed at a radius (r2) from the central axis 80. In an embodiment, two radii (r2) can be equal to the second diameter (d2), i.e. 2(r2)=(d2). The finger 120 can extend into the catheter lumen 92 before an axial force is required to urge the catheter 90 over the stem 100. Advantageously, the finger 120 can guide the catheter 90 on to the stem 100 as well as providing columnar support to the catheter 90 as the catheter 90 is urged over the stem 100 mitigating kinking or collapsing of the catheter lumen 92. Advantageously, a stem 100 including a tip structure 104 as described herein can be coupled with a greater range of catheter sizes increasing the versatility of the stem and reducing the product inventory required to be transported and stored by a user, reducing associated costs.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A stem for providing fluid communication between an access port and a catheter, the stem comprising:
an elongate body defining a body lumen, a distal opening of the body lumen having a first diameter; and
a tip structure extending distally from the distal opening and comprising:
a tip lumen having a second diameter smaller than the first diameter; and
a plurality of slots extending from a distal end of the tip structure to the distal opening of the body lumen.

2. The stem according to claim 1, wherein the plurality of slots define a plurality of fingers.

3. The stem according to claim 2, wherein the plurality of fingers taper from a first wall thickness at a proximal end to a second wall thickness, less than the first wall thickness, at a distal end.

4. The stem according to claim 1, wherein the tip structure comprises an outer surface that tapers distally from a first larger diameter to a second smaller diameter.

5. The stem according to claim 1, wherein the elongate body comprises a circumferential ridge disposed along an outer surface of the elongate body.

6. The stem according to claim 1, wherein the stem is coupled to the access port with one of an interference fit, press-fit, or snap-fit engagement.

7. The stem according to claim 1, wherein the slot extends through an arc distance of less than 350°.

8. The stem according to claim 2, wherein a finger of the plurality of fingers extends radially through an arc distance of less than 350º.

9. A stem for providing fluid communication between a medical device and a catheter, comprising:
a body defining a lumen extending between a proximal opening and a distal opening, the distal opening defining a first diameter;
a tip structure disposed at a distal end of the body, comprising:
one or more fingers extending radially inwards and distally from the distal opening, an inner surface of a first finger of the one or more fingers extending along an axis disposed radially inward from the distal opening.

10. The stem according to claim 9, wherein the first finger extends radially through an arc distance of less than 350°.

11. The stem according to claim 9, wherein the finger includes a proximal portion extending from the distal opening radially inward and distally, and a distal portion supported by the proximal portion and extending distally therefrom.

12. The stem according to claim 11, wherein an inner surface of the distal portion extends parallel to the longitudinal axis and an outer surface of the distal portion extends at an angle relative to the longitudinal axis.

13. The stem according to claim 9, further including a second finger of the plurality of fingers and disposed at a different radial position about the axis of the lumen, an inner surface of the first finger and an inner surface of the second finger defining a second diameter that is less than the first diameter.

14. The stem according to claim 13, wherein an outer surface of the first finger and an outer surface of the second finger defines a tapered outer profile extending from the second diameter to the first diameter.

15. The stem according to claim 13, wherein the first finger and the second finger define a slot extending longitudinally therebetween to the distal opening.

16. The stem according to claim 15, wherein the slot extends radially through an arc distance of less than 350°.

17. A method of coupling a catheter with a medical device, comprising:
providing a stem formed of a rigid material and defining a lumen extending between a distal opening and a proximal opening, the distal opening defining a first diameter, the stem having a distal tip structure including two or more fingers extending longitudinally distally from the distal opening, distal tip of the two or more fingers co-operating to form a second diameter, less than the first diameter;
providing a catheter formed of a compliant material and defining a lumen having a third diameter that is larger than the second diameter;
urging the tip structure into the catheter lumen; and
elastically deforming the catheter from the third diameter to the first diameter.

18. The method according to claim 17, wherein the third diameter of the catheter lumen is equal to or less than the first diameter of the distal opening.

19. The method according to claim 17, wherein a finger of the two or more fingers includes a proximal portion extending radially inward from the distal opening, and a distal portion extending longitudinally distally from the proximal portion and defining the inner surface.

20. The method according to claim 17, wherein an outer surface of the two or more fingers defines a tapered profile extending proximally from the second diameter to the first diameter.

21. The method according to claim 17, wherein the two or more fingers define a slot extending longitudinally therebetween from a distal tip of the tip structure and the distal opening.
